(19) United States
(12) Patent Application Publication
(10) Pub. No.: US 2019/0275095 A1
(43) Pub. Date: Sep. 12, 2019

(54) Topical Salves and Lotions, and Methods

(71) Applicants: Christie Sievers Spencer, Boulder, CO (US); Robert E. Sievers, Boulder, CO (US)

(72) Inventors: Christie Sievers Spencer, Boulder, CO (US); Robert E. Sievers, Boulder, CO (US)

(21) Appl. No.: 16/298,904

(22) Filed: Mar. 11, 2019

Related U.S. Application Data
(60) Provisional application No. 62/641,711, filed on Mar. 12, 2018.

(51) Int. Cl. A61K 36/185; A61K 47/44; A61K 47/46; A61K 9/06; A61K 9/00; A61P 25/04

(52) U.S. Cl. CPC A61K 36/185; A61K 47/44; A61P 25/04; A61K 9/06; A61K 9/0014; A61K 47/46

(57) ABSTRACT

A salve for topical application comprises beeswax, coconut oil and at least one cannabinoid extract. A lotion for topical application comprises grapeseed oil, sweet almond oil, and at least one cannabinoid extract. Methods of alleviating pain, for example, associated with at least one of osteoarthritis, restless leg syndrome, carpal tunnel syndrome, localized spasticity, pinched nerve, fibromyalgia, neuropathy, muscle pain, lymphedema, tissue injury involving swelling, and damaged, dry, or cracking skin, in an individual, comprise topically applying the salve or the lotion to the skin of the individual.

### RELATED APPLICATION

The present application claims priority under 35 U.S.C. 119 of U.S. Application No. 62/641,711 filed Mar. 12, 2018.

### FIELD OF THE INVENTION

The present invention relates to topical salves and lotions which employ one or more cannabinoids in combination with natural ingredients, and to methods for alleviating various conditions by topical application of such compositions.

### BACKGROUND OF THE INVENTION

Active pharmaceutical ingredients (APIs) extracted from the Cannabis plant, commonly referred to as cannabinoids, have been disclosed for various medical uses, including, but not limited to, treatment of epileptic seizures, treatment of cancer, reducing adverse effects of cancer treatments, pain management, and treatment of auto-immune disorders. Often times, cannabinoids are formulated in oral dosage forms which facilitate use for particular therapeutic treatments. However, in some instances, other dosage forms are desirable.

### SUMMARY OF THE INVENTION

Accordingly, it is an object of the present invention to provide salves and lotions for topical application and to provide methods for alleviating alleviating various conditions by topical application of such compositions.

In one embodiment, the invention is directed to salves for topical application. The salves comprise beeswax, coconut oil and at least one cannabinoid extract.

In another embodiment, the invention is directed to lotions for topical application. The lotions comprise grapeseed oil, sweet almond oil, and and at least one cannabinoid extract.

In another embodiment, the invention is directed to methods of alleviating pain, for example, pain associated with at least one of osteoarthritis, restless leg syndrome, fibromyalgia, neuropathy, muscle pain, tissue injury involving swelling, or damaged, dry, or cracking skin, in an individual. The methods comprise topically applying the salve or lotion to the skin of the individual.

The salves and lotions provide a convenient manner in which to alleviate pain in an individual by topical application. These and additional embodiments and advantages will be more fully understood in view of the following detailed description.

### DETAILED DESCRIPTION

The salves and lotions according to the invention are intended for topical application and, more specifically, for application to the skin of an individual.

The salves and lotions comprise a combination of naturally-derived ingredients and may be used, inter alia, to alleviate various conditions, including pain, muscle restlessness such as leg restlessness, particularly in older patients, stress, and the like.

In one embodiment, the salves comprise a base formed of beeswax and coconut oil. The beeswax and coconut oil are employed in amounts sufficient to provide the salve with a semi-solid constancy at room temperature. In specific embodiments, the salves comprise from about 3 to about 10 weight percent beeswax, and from about 90 to about 97 weight percent coconut oil, based on the combined weight of the beeswax and coconut oil.

In one embodiment, the lotions comprise grapeseed oil and sweet almond oil. The lotions may comprise these oils in any desired amounts. For example, the lotions may comprise from about 10 to about 90 weight percent grapeseed oil and from about 90 to about 10 weight percent sweet almond oil, based on the combination of the oils. In another embodiment, the lotions comprise from about 25 to about 75 weight percent grapeseed oil and from about 75 to about 25 weight percent sweet almond oil, based on the combination of the oils.

The salves and lotions further include at least one cannabinoid extract, for example, an extract of at least one of cannabidiol (CBD), Δ9-tetrahydrocannabinol (THC), tetrahydrocannabinolic acid (THCA), cannabidiolic acid (CBDA), cannabinol (CBN), cannabigerol (CBG), cannabichromene (CBC), cannabitriol (CBT), or combinations of two or more thereof. In more specific embodiments, the cannabinoid extract comprises CBD; THC; THCA; CBD and THC; CBD and CBN; or CBD, THC, and CBN. In a more specific embodiment, the cannabinoid extract comprises CBD. The extract may be prepared according to methods well known in the art. In one embodiment, the extract is prepared according to a method as disclosed in the Sievers et al U.S. application Ser. No. 15/017,384, which is incorporated herein by reference. Thus, such cannabinoid extracts may be prepared by extraction with a hydrocarbon solvent, and/or carbon dioxide, for example high pressure carbon dioxide extraction, and subjected to various purification methods, for example silica column purification, high pressure liquid chromatography purification, activated carbon, and the like. In specific embodiments, the cannabinoid extract comprises at least about 75 weight percent cannabinoid, at least about 90 weight percent cannabinoid, or, more specifically, at least about 95 weight percent cannabinoid.

The salves and lotions comprise an effective amount of the cannabinoid(s) to achieve a desired effect. In one embodiment, the salves and lotions comprise from about 0.01 to about 10 weight percent cannabinoid extract. In more specific embodiments, the salves and lotions comprise from about 0.01 to about 1 weight percent cannabinoid extract. In a more specific embodiment, the salves and lotions comprise from about 25 to about 75 mg CBD, and/or from about 1 to about 75 mg THC, per ounce of product. In additional embodiments, the salves and lotions comprise a micro-dosing or nano-dosing amount of cannabinoid extract, wherein the salves and lotions comprise from about 0.0001 to less than 0.01 weight percent cannabinoid extract. In a more specific micro-dosing or nano-dosing embodiment, the salves and lotions comprise from about 1 to about 10 mg CBD, and/or from about 0.01 to less than 1 mg THC or CBN, per ounce of product.

The salves and lotions according to the invention may further comprise one or more additional natural ingredients. Examples of such ingredients include, but are not limited to, essential oils, for example, peppermint oil, lavender oil, rosemary extract, eucalyptus oil, and hempseed oil, menthol crystals, sunflower lecithin, cabbage extract (for example, in sunflower oil), cayenne pepper extract, ginger extract, frankincense, rosehip oil, and the like. Such ingredients may, for example, be added in an amount of from about 0.001 to about 1 weight percent, or, more specifically, from about 0.001 to about 0.1 weight percent.

In a specific embodiment, a salve for topical application according to the invention comprises beeswax, coconut oil, and from about 0.01 to about 10 weight percent cannabidiol (CBD) extract, wherein the CBD extract comprises at least about 75 weight percent CBD. In more specific embodiments, such salves further comprise rosemary extract and sunflower lecithin. In still more specific embodiments, such salves further comprise at least one essential oil, for example, at least one of peppermint oil, lavender oil, eucalyptus oil, and hempseed oil.

In a specific embodiment, a lotion for topical application according to the invention comprises grapeseed oil, sweet almond oil, and from about 0.01 to about 10 weight percent cannabidiol (CBD) extract, wherein the CBD extract comprises at least about 75 weight percent CBD. In more specific embodiments, such lotions further comprise rosemary extract and sunflower lecithin. In still more specific embodiments, such salves further comprise at least one essential oil, for example, at least one of peppermint oil, lavender oil, eucalyptus oil, and hempseed oil.

The salves may be prepared by heating the beeswax to allow incorporation of the remaining ingredients. The heating temperature must be carefully controlled to avoid decomposition of the cannabinoid in the extract included in the particular salve. In one embodiment, a cannabidiolic acid (CBDA) extract is heated above about 135° C., resulting in decomposition of the carboxylic acid and provision of cannabidiol (CBD) in the salve composition. Cooling of the composition after addition of all desired ingredients allows the composition to solidify to the desired semi-solid form which facilitates topical application.

The lotions may be prepared by mixing the grapeseed oil, sweet almond oil, and the remaining ingredients. The lotions may be dispensed via a pump container, or from a bottle with a rolling ball top, or via any other means known in the art for dispensing liquids.

The salves and lotions of the invention are particularly advantageous for use in alleviating pain in an individual by topically applying the salve or lotion to the skin of the individual. Cutaneous delivery of the cannabinoid by topical application of a salve or lotion according to the invention allows low doses, including micro-dosed amounts or nano-dosed amounts, of the cannabinoid to enter the body and interact with the receptors in the endocannabinoid system. Topically applying, for example, CBD, in the described salve or lotion formulations has been experimentally demonstrated to relieve pain in test subjects. In one embodiment, the salve or lotion is topically applied to the skin of an individual at a location proximate to an area where pain is sensed. For example, the salves and lotions may be so employed in methods for alleviating pain associated with at least one of osteoarthritis, restless leg syndrome, carpal tunnel syndrome, localized spasticity, pinched nerve, fibromyalgia, neuropathy, muscle pain, and/or tissue injury involving swelling, in an individual, by topically applying the salve to the skin of the individual. In additional embodiments, the salves and lotions of the invention are suitable for use in a method of alleviating pain associated with damaged, dry, or cracking skin, in an individual by topically applying the salve or lotion to the affected skin of the individual. Further, the the salves and lotions may be applied to alleviate symptoms associated with psoriasis, to reduce acne and/or acne scarring, and/or to reduce post-operative skin cancer scarring. In additional embodiments, the salves and lotions of the invention are suitable for use in alleviating symptoms associated with Lymphedema and localized muscular spasticity.

In specific embodiments, neuropathy and restless leg syndrome can be treated transdermally with a salve according to the invention. In one experiment, a patient (male, 80+ years, with unremediated atrial fibrillation, compounded by periodic stabbing acute toe pain) obtained significant alleviation of pain from gout attacks by periodic daily topical application to toes and legs of a CBD-containing salve according to the invention. This specific patient was also undergoing treatment with allopurinol (400-600 milligrams daily for gout) and metoprolol beta blocker (50 milligrams per day to lower increased heart rate and breathlessness from atrial fibrillation) taken orally. The transdermal application of the salve noticeably improved pain alleviation as compared with the oral administration of allopurinol and metoprolol beta blocker in the absence of the salve application. Other diseases such as Parkinson's disease are similarly suitable for supplementing traditional medicament administration with salve or lotion topical application according to the invention.

In additional embodiments, the salves and lotions may be used for calming muscle restlessness, for example, leg restlessness which is often encountered in older patients, and calming pets in stressful situations, for example, pets agitated by thunderstorms.

The following examples demonstrate embodiments of the invention.

### Example 1

The following ingredients are heated and mixed until all ingredients are homogeneously combined: beeswax, cannabidiol extract, coconut oil, peppermint oil, lavender oil, rosemary extract, menthol crystals, eucalyptus oil, and sunflower lecithin. The mixture is cooled to room temperature to a semi-solid form and applied topically as needed for pain relief from osteoarthritis, restless leg syndrome, carpal tunnel syndrome, fibromyalgia pain or neuropathic pain.

### Example 2

The following ingredients are heated and mixed until all ingredients are homogeneously combined: beeswax, cannabidiol extract, coconut oil, cabbage extract in sunflower oil, rosemary extract, menthol crystals, eucalyptus oil, and sunflower lecithin. The mixture is cooled to room temperature to a semi-solid form and applied topically to reduce pain and tissue swelling from osteoarthritis and tissue injury involving swelling. In Chinese cultures, cabbage has been used to reduce swelling in damaged tissue. This salve formulation therefore combines the attributes of cabbage with the benefits of CBD.

### Example 3

The following ingredients are heated and mixed until all ingredients are homogeneously combined: beeswax, cannabidiol extract, coconut oil, peppermint oil, cayenne pepper extract, ginger extract, rosemary extract, menthol crystals, eucalyptus oil and sunflower lecithin. The mixture is cooled to room temperature to a semi-solid form and applied topically to alleviate muscle pain caused by over exertion or injury.

### Example 4

The following ingredients are heated and mixed until all ingredients are homogeneously combined: beeswax, cannabidiol extract, coconut oil, peppermint oil, lavender oil, rosemary extract, hempseed oil, and sunflower lecithin. Cool to room temperature. The mixture is cooled to room temperature to a semi-solid form and applied topically to damaged, dry, or cracking skin.

### Example 5

The following ingredients and mixed until they are homogeneously combined: grapeseed oil, sweet almond oil, peppermint oil, lavender oil, rosemary extract, cannabinoid extract, and sunflower lecithin. The cannabinoid extract comprises CBD, CBD and THC, CBD and CBN, or CBD, THC and CBN. The mixture is applied topically as needed for pain relief from osteoarthritis, restless leg syndrome, carpal tunnel syndrome, fibromyalgia, or neuropathy, or for stress relief.

The various examples and embodiments described herein are exemplary only and are not to be construed as limiting the scope of the invention defined by the following claims. Throughout this specification, when a range of conditions or a group of substances is defined with respect to a particular characteristic (e.g., temperature, pressure, amounts, and the like) of the present invention, the present invention relates to and explicitly incorporates every specific member and combination of subranges or subgroups therein. Any specified range or group is to be understood as a shorthand way of referring to every member of a range or group individually as well as every possible subrange and subgroup encompassed therein; and similarly with respect to any subranges or subgroups therein.

What is claimed is:

1. A salve for topical application, comprising beeswax, coconut oil and at least one cannabinoid extract.

2. The salve of claim 1, comprising the beeswax and coconut oil in amounts sufficient to provide a semi-solid constancy at room temperature.

3. The salve of claim 1, comprising from about 0.01 to about 10 weight percent cannabinoid extract.

4. The salve of claim 3, wherein the cannabinoid extract comprises at least about 75 weight percent cannabinoid.

5. The salve of claim 3, wherein the cannabinoid extract comprises at least about 90 weight percent cannabinoid.

6. The salve of claim 3, wherein the cannabinoid extract comprises at least about 95 weight percent cannabinoid.

7. The salve of claim 1, wherein the cannabinoid extract comprises cannabidiol (CBD), Δ9-tetrahydrocannabinol (THC), tetrahydrocannabinolic acid (THCA), cannabidiolic acid (CBDA), cannabinol (CBN), cannabigerol (CBG), cannabichromene (CBC), cannabitriol (CBT), or combinations of two or more thereof.

8. The salve of claim 1, wherein the cannabinoid extract comprises CBD; THC; THCA; CBD and THC; CBD and CBN; or CBD, THC, and CBN.

9. The salve of claim 1, further comprising one or more of peppermint oil, lavender oil, rosemary extract, menthol crystals, eucalyptus oil, sunflower lecithin, cabbage extract, cayenne pepper extract, ginger extract, frankincense, rosehip oil, and hempseed oil.

10. A salve for topical application, comprising beeswax, coconut oil, and from about 0.01 to about 10 weight percent cannabidiol (CBD) extract, wherein the CBD extract comprises at least about 75 weight percent CBD.

11. The salve of claim 10, further comprising rosemary extract and sunflower lecithin.

12. The salve of claim 10, further comprising at least one of peppermint oil, lavender oil, eucalyptus oil, and hempseed oil.

13. A method of alleviating pain in an individual, comprising topically applying the salve of claim 1 to the skin of the individual.

14. The method of claim 13, wherein the pain is associated with at least one of osteoarthritis, restless leg syndrome, carpal tunnel syndrome, localized spasticity, pinched nerve, fibromyalgia, neuropathy, muscle pain, lymphedema, tissue injury involving swelling, and damaged, dry, or cracking skin, in the individual.

15. A lotion for topical application, comprising grapeseed oil, sweet almond oil, and at least one cannabinoid extract.

16. The lotion of claim 15, comprising from about 0.01 to about 10 weight percent cannabinoid extract.

17. The salve of claim 15, wherein the cannabinoid extract comprises at least about 75 weight percent cannabinoid.

18. The lotion of claim 15, further comprising one or more of peppermint oil, lavender oil, rosemary extract, menthol crystals, eucalyptus oil, sunflower lecithin, cabbage extract, cayenne pepper extract, ginger extract, frankincense, rosehip oil, and hempseed oil.

19. A method of alleviating pain in an individual, comprising topically applying the lotion of claim 15 to the skin of the individual.

20. The method of claim 15, wherein the pain is associated with at least one of osteoarthritis, restless leg syndrome, carpal tunnel syndrome, localized spasticity, pinched nerve, fibromyalgia, neuropathy, muscle pain, lymphedema, tissue injury involving swelling, and damaged, dry, or cracking skin, in the individual.
